(19) United States
(12) Patent Application Publication
(10) Pub. No.: US 2026/0072113 A1
(43) Pub. Date: Mar. 12, 2026

(54) SYSTEMS AND METHODS REGARDING LONGITUDINAL GRASP MRI

(71) Applicant: NEW YORK UNIVERSITY, New York, NY (US)

(72) Inventors: Douglas Kondziolka, New York, NY (US); Kai Tobias Block, New York, NY (US); Girish Fatterpekar, New York, NY (US)

(73) Assignee: NEW YORK UNIVERSITY, New York, NY (US)

(21) Appl. No.: 18/868,122

(22) PCT Filed: May 22, 2023
(86) PCT No.: PCT/US2023/023129

§ 371 Date: Nov. 21, 2024

Related U.S. Application Data
(60) Provisional application No. 63/344,725, filed on May 23, 2022.

(51) Int. Cl. G01R 33/561; A61B 5/055; G01R 33/48; G01R 33/56

(52) U.S. Cl. CPC G01R 33/5611; A61B 5/055; G01R 33/4824; G01R 33/5608

(57) ABSTRACT

A method can include receiving, by at least one processor, a magnetic resonance dataset comprising at least one scan. The method can include performing, by the at least one processor, golden-angle radial sparse parallel imaging on the magnetic resonance dataset to output one or more images. The method can include identifying, by the at least one processor, at least one region of interest in the one or more images, the at least one region of interest corresponding to at least one of tumor progression or radiation effects.

### CROSS-REFERENCE TO RELATED APPLICATIONS

The present application claims the benefit of priority to U.S. Provisional Application No. 63/344,725, filed May 23, 2022, the entire contents of which are incorporated herein by reference.

### TECHNICAL FIELD

The present disclosure relates generally to MRI and more specifically to distinguishing brain metastasis progression from radiation effects after stereotactic radiosurgery using longitudinal golden-angle radial sparse parallel (“GRASP”) dynamic contrast-enhanced MRI.

### BACKGROUND

Health care professionals can use radiation to treat both benign and malignant tumors. In general, health care professionals can determine the amount of radiation (the “dose”) to be used based on several different factors. These factors can include prior experience, available data about clinical outcomes for tumor responses, tumor volume, safety data regarding radiation received by adjacent body tissue, other available treatment options that could affect the use of higher or lower doses of radiation, and pathological information on tumor cell appearance and the rate of cell division (i.e., mitoses), which can require obtaining a biopsy tissue sample.

### SUMMARY

Systems and methods for differentiating brain-metastasis progression from radiation effects or necrosis. GRASP dynamic contrast-enhanced MRI provides high spatial and temporal resolution to analyze tissue enhancement. Methods and systems for utilizing GRASP contrast enhanced MRI enables distinction between metastasis progression and radiation necrosis.

At least one aspect is directed to a method. The method can include receiving, by at least one processor, a magnetic resonance dataset comprising at least one scan. The method can include performing, by the at least one processor, golden-angle radial sparse parallel imaging on the magnetic resonance dataset to output one or more images. The method can include identifying, by the at least one processor, at least one region of interest in the one or more images, the at least one region of interest corresponding to at least one of tumor progression or radiation effects.

At least one aspect is direct to a system. The system can include at least one processor. The system can include a memory, with computer code instructions stored thereon. The computer code instructions, when executed by the at least one processor, can cause the at least one processor to receive a magnetic resonance dataset comprising at least one scan. The computer code instructions, when executed by the at least one processor, can cause the at least one processor to perform golden-angle radial sparse parallel (GRASP) imaging on the magnetic resonance dataset to output one or more images. The computer code instructions, when executed by the at least one processor, can cause the at least one processor to identify at least one region of interest in the one or more images, the at least one region of interest corresponding to at least one of tumor progression or radiation effects.

### BRIEF DESCRIPTION OF THE FIGURES

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

FIGS. 1A-1D illustrate a hotspot method of selecting regions of interest, according to an embodiment.

FIGS. 2A-2H illustrate progressive metastasis with signal-time curves, according to an embodiment.

FIGS. 3A-3G illustrate radiation necrosis with signal-time curves, according to an embodiment.

FIGS. 4A-4F illustrate a responsive control tumor with signal-time curves, according to an embodiment.

FIGS. 5A-5D are boxplots of normalized wash-in slopes, according to an embodiment.

FIGS. 5E-5H are boxplots of normalized wash-out slopes, according to an embodiment.

FIG. 6 illustrates ROC curves for the differentiation of tumor progression and radiation necrosis, according to an embodiment.

FIG. 7 illustrates a method of magnetic resonance imaging, according to an embodiment.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Following below are more detailed descriptions of various concepts related to, and implementations of, methods, apparatuses, and systems for longitudinal GRASP MRI. The various concepts introduced above and discussed in greater detail below may be implemented in any of a number of ways, as the described concepts are not limited to any particular manner of implementation. Examples of specific implementations and applications are provided primarily for illustrative purposes.

Many forms of radiation delivery can be image-guided. Available imaging approaches can include magnetic resonance imaging (“MRI”), computed tomography (“CT”), x-ray imaging, and positron emission tomography (“PET”), among others. Generally speaking, the images acquired via the aforementioned techniques can typically be obtained on or shortly before the day that the radiation dose is administered to the patient.

Stereotactic radiosurgery (“SRS”) can have value in the care of various types of intracranial tumors including brain metastases in particular. Radiation-related changes following any type of brain irradiation can be detected on routine follow-up MRI scans. Differentiating tumor progression (“TP”) from adverse radiation effects, which can include radiation necrosis (“RN”), can be an important but challenging diagnostic dilemma for clinicians, as each implies a different therapeutic approach.

MRI can often be inadequate in distinguishing tumor progression from radiation effects (e.g., radiation necrosis). Advanced imaging techniques, such as dynamic contrast-enhanced (“DCE”) MRI and dynamic-susceptibility contrast (“DSC”) MRI, can be used to provide complementary information in this diagnostic dilemma. However, so far no technique has been adopted as the standard of care due to inconsistent results reported in the literature, technical limitations (i.e., brain location, lesion size, intralesional hemorrhage), and variability in the utilized multicompartmental physiologic models. A recent development in DCE-MRI is golden-angle radial sparse parallel (“GRASP”) imaging, which is characterized by high spatial and temporal resolution, robustness to motion and pulsation artifacts, and ease of use. GRASP has already shown promising results in preoperative mapping of the pituitary gland in patients with macroadenomas, differentiating between head and neck schwannoma from paraganglioma, distinguishing different types of salivary gland tumors, as well as in other applications in prostate, cardiac, liver, and breast imaging.

There remains a need for methods and systems to improve upon existing available means, such as by the proposed GRASP imaging, to help differentiate tumor progression (e.g., metastasis progression) from radiation necrosis.

One embodiment of the present disclosure relates to systems and method for using GRASP MRI, specifically such as Longitudinal GRASP DCE-MRI helps to differentiate brain-metastasis progression from radiation necrosis, using the normalized wash-in slope as a clinically usable, model-free measure of the tissue enhancement pattern.

A recent development in DCE-MRI is GRASP imaging, which is characterized by high spatial and temporal resolution, robustness to motion and pulsation artifacts, and ease of use. U.S. patent application Ser. No. 17/676,487, which is incorporated herein by reference, describes an embodiment of GRASP. GRASP has already shown promising results in preoperative mapping of the pituitary gland in patients with macroadenomas, differentiating between head and neck schwannoma from paraganglioma, distinguishing different types of salivary gland tumors, as well as in other applications in prostate, cardiac, liver, and breast imaging. However, described herein are systems and methods for distinguishing between tumor cells and cells exhibiting the effects of treatment, such as radiation treatment.

In one embodiment, a method includes improved tumor imaging through the use of dynamic, quantitative assessment of intravenous contrast uptake. Specific profiles for different tumors and therapeutic responses can be created which can allow for rapid, safe, and effective diagnoses specifically aimed at improving patient care.

In one embodiment, GRASP MRI is utilized to determine the tissue enhancement characteristics of untreated tumors and subsequently their response characteristics associated with different forms of therapy (e.g., radiation based, drug based, other treatments). In one embodiment, permeability characteristics evaluated will be indirectly evaluated utilizing enhancement-time curves, wash-in slope, wash-out, and area under curve (“AUC”) measures. In one embodiment, the system and methods utilize information from historical databases or may utilize predictive information based upon AI. For example, in one implementation historical data from similar tumors may be used to train machine learning devices, enabling an AI informed analytical approach for characterization of new patient imaging data. Further, the methods and system may utilize subjective patient information, such as the patients' own tumor and brain region derived information, rather than information derived from the experience obtained in the care of other patients. Specifically in one embodiment, a patient with a tumor is imaged, the dynamic response characteristics of the tumor are measured, and this data is used to create a profile for the tumor in its native untreated state, or after treatment.

The use of GRASP in the described embodiment, can enable a more accurate determination of the nature of a tumor based on its dynamic vascular characteristics in order to separate the natural state of different tumors, the treatment response of the tumor, identification of recurrence, and identification of the treatment response (which leads to tumor or regional tissue injury).

TP and RN can exhibit quantitative enhancement differences on dynamic GRASP imaging. Although the physiologic basis for contrast-induced T1-weighted signal-intensity changes remains incompletely understood, TP can be characterized by a rapid influx of contrast (wash-in) due to a viable and hyperdynamic microvasculature, as opposed to RN, which can have a significantly slower wash-in due to granulation tissue and radiation vasculopathy. The experimental data study examined embodiments of brain-metastases patient registry in those patients who underwent SRS and had multiple imaging examinations with GRASP.

### Experimental Study

### Patient Population

An IRB-approved, HIPAA-compliant retrospective study was undertaken. The study included patients with intra-axial brain metastases managed with gamma-knife SRS who had GRASP before and at least once after SRS. Patients were identified with pathologically confirmed tumor progression from post-SRS resected lesions and patients who were diagnosed with RN, based on either surgically resected tissue with no signs of tumor or on lesion resolution on imaging follow-up. A separate group of non-small lung cancer (“NSCLC”) patients with brain metastases ≥1 cm in diameter that showed favorable response with tumor control and without RN on subsequent imaging were also identified as a reference.

Radiosurgery was performed using the Leksell Gamma Knife Perfexion or Icon model. The first outcomes assessments were scheduled at 2 months and then at every 3 months for the first two years. Imaging was obtained every 4 months from years 2-4, and subsequently at 6-month intervals if there were no new tumors or concerning recurrences. Imaging outcomes were classified using the Response Evaluation Criteria in Solid Tumors (“RECIST”) as “progressive disease (“PD”),” in comparison to tumor control: either “partial response (“PR”),” “complete response (“CR”),” or “stable disease (“SD”).” Any adverse radiation effects (“ARE”) or RN, either asymptomatic or symptomatic, were documented. Peritumoral patchy enhancement with a mismatch on the long relaxation time images were coded as an inflammatory change and then evaluated with GRASP MRI.

### GRASP Imaging

Dynamic GRASP imaging has been described, including in U.S. patent application Ser. No. 17/676,487, incorporated herein by reference. GRASP is a 3D gradient-echo (“GRE”) sequence based on continuous radial stack-of-stars k-space sampling according to the golden-angle scheme (angular increment of 111.25°). After data acquisition, consecutive spokes in k-space are binned into temporal frames. The number of spokes per frame can be selected arbitrarily, allowing to customize the obtained temporal resolution. The data are reconstructed using parallel-imaging and compressed-sensing principles to produce artifact-free images with high spatial resolution.

GRASP imaging was obtained using MRI systems with field strength of 1.5 T or 3 T (Siemens Healthcare, Erlangen, Germany). Acquisition parameters included: TR=3.45-6.86 ms, TE=1.69-2.1 ms, flip angle=9-12°, matrix size=256×256, 1 mm isotropic spatial resolution. After a delay of 20 seconds relative to the sequence start, a single 0.1 mL/kg gadobutrol (Gadavist, Bayer Schering Pharma, Berlin, Germany) bolus was administered intravenously at the rate of 4 mL/s. Time of acquisition for the GRASP sequence was 3-5 minutes with a median of 480 total spokes acquired (range, 400-897 total spokes). Images were reconstructed with 13 or 40 spokes per frame, resulting in a median temporal resolution of 13.93 seconds per frame (range, 3.75-20.14 seconds per frame).

### GRASP Analysis

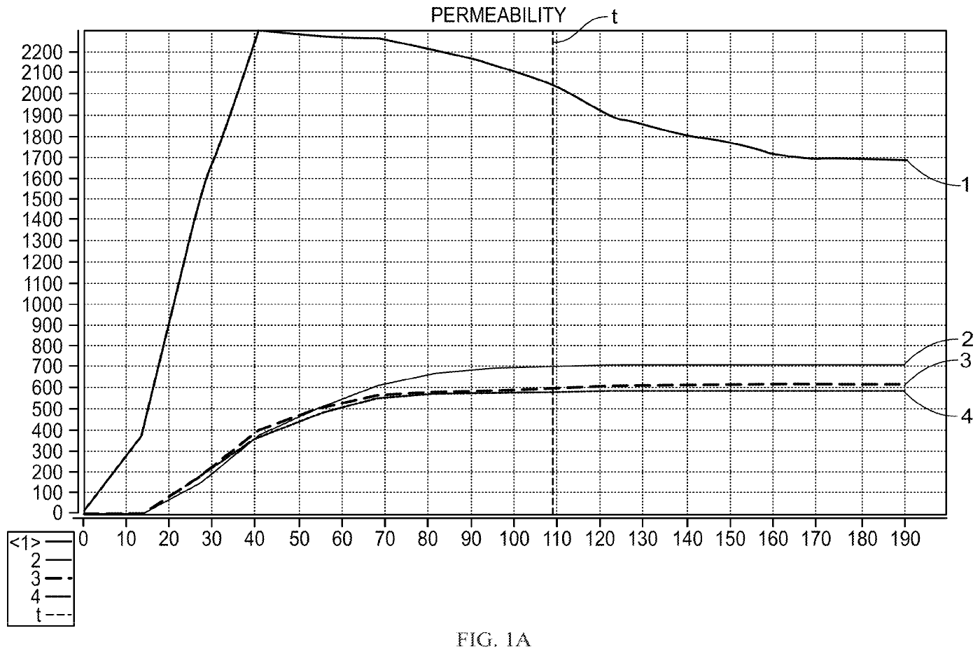

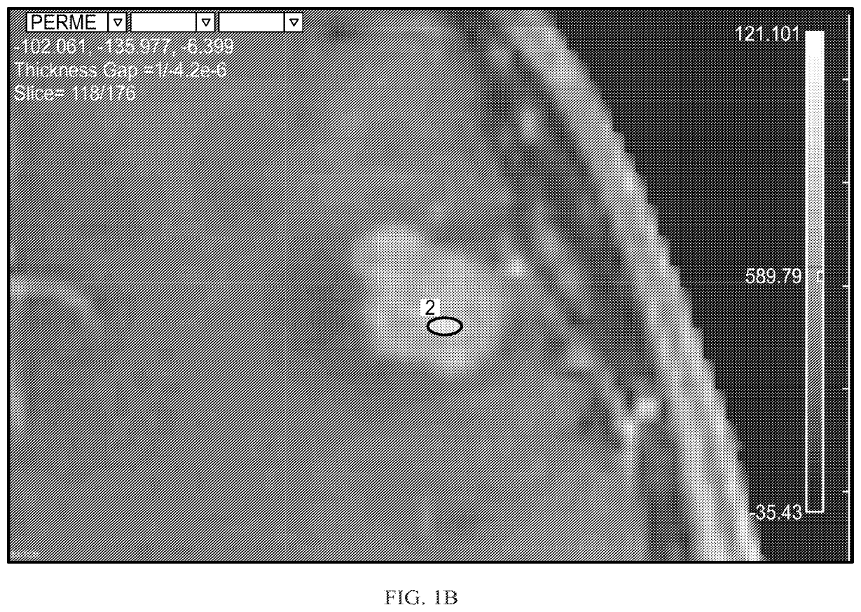

Analysis was limited to the first 100 seconds of acquisition. For each scan, 3 non-overlapping ROIs in the maximally enhancing component of the tumor that received SRS and a control ROI in the superior sagittal sinus were drawn using the software Olea Sphere 3.0 (Olea Medical, La Ciotat, France) under the guidance of a board-certified neuroradiologist with 15 years post-fellowship experience (G.F.). Maximally enhancing components were identified using the dynamic GRASP images as well as color-coded parametric maps of peak enhancement and area under the signal-time curve automatically generated by Olea Sphere (FIGS. 1A-1D). Vessels and cystic/necrotic components were avoided. Signal-time curves were extracted from each ROI, and the slopes of the curves during wash-in (period of maximally rapid increasing superior sagittal sinus signal) and wash-out (period of monotonically decreasing signal intensity after peak superior sagittal sinus enhancement) were calculated. Tumor ROIs' wash-in slope and wash-out slope were normalized to the superior sagittal sinus as an internal control on each scan. For each scan, normalized wash-in and wash-out slope were averaged across all 3 tumor ROIs. Mean normalized wash-in and wash-out slope were compared between groups.

FIGS. 1A-1D illustrate a hotspot method of selecting regions of interest (“ROI”) on dynamic contrast-enhanced GRASP imaging using the Olea Sphere software. The signal-time curves (FIG. 1A) correspond to the ROIs drawn on the GRASP image (FIG. 1B) and color parametric maps of area under the signal-time curve (FIG. 1C) and peak enhancement (FIG. 1D).

### Statistical Analysis

Statistical analysis was performed in Matlab (MathWorks, Natick, Massachusetts). Categorical variables were compared by Pearson's χ² test, while continuous variables were compared by the student's t-test with significance level p<0.05. Receiver-operating characteristic (“ROC”) analysis was performed to calculate AUC. Youden's index was calculated to determine the diagnostic performance of different thresholds for differentiating TP and RN.

### Results

### Clinical

Clinical, demographic, and SRS parameters of the different groups are presented in Table 1. Forty-eight patients were included: 16 with TP, 16 with RN, and 16 with tumor control. Mean age of the entire study population was 59±15 years, and 40% were male. Seventeen patients underwent surgical resection of their lesion, with 16 showing progression/recurrence and one showing RN on pathology. The remaining 15 RN patients had imaging and clinical outcomes consistent with RN. Following SRS, the studied volumes were larger in 63% of either the tumor progression or RN groups (10 patients in each group). Maximal lesion enlargement in the tumor progression and RN groups was 11±18 cc and 3±6 cc (p=0.220), occurring at 6±6 months and 13±10 months post-SRS, respectively. Among 6 patients in each group (37%), the entire volume of each lesion became smaller after SRS, yet there was a segmental growing nodularity that raised suspicion for either progression or radiation effects.

TABLE 1
Demographic, clinical and radiosurgery parameters in the tumor control (n =
16), tumor progression (n = 16) and radiation necrosis (n = 16) groups.
P values represent comparisons between the tumor progression (“TP”) and RN groups.

| | | Tumor | Radiation | |
|---|---|---|---|---|
| | Tumor control | progression | Necrosis | P Value |
| Parameter | (n = 16) | (n = 16) | (n = 16) | (TP vs RN) |
| Age ± SD | 68 ± 12 | 51 ± 15 | 58 ± 14 | 0.176 |
| Sex, male, % | 8, 50% | 7, 47% | 4, 24% | 0.169 |
| KPS, median, range | 90, 70-90 | 90, 80-100 | 90, 80-90 | 0.07 |
| Tumor volume | 1.96 ± 2.02 | 5.02 ± 8.4 | 3.19 ± 4.45 | 0.439 |
| Marginal dose | 18, 16-20 | 19, 15-20 | 18, 13-20 | 0.385 |
| Minimal Dose | 15, 10-18 | 15, 11-18 | 14, 9-21 | 0.692 |
| Maximal Dose | 36, 20-40 | 38, 30-40 | 36, 21-40 | 0.210 |
| Mean Dose | 25, 19-30 | 26, 20-30 | 25, 18-32 | 0.823 |
| 12 Gy | 5.2 ± 4.6 | 10.4 ± 14.7 | 7.1 ± 8.3 | 0.428 |
| Immunotherapy | 9, 56% | 6, 40% | 7, 41% | 0.946 |
| Targeted Treatment | 7, 44% | 5, 33% | 8, 47% | 0.430 |
| Chemotherapy | 3, 19% | 5, 33% | 9, 53% | 0.265 |
| WBRT | 0 | 1, 7% | 2, 12% | 0.621 |

SD: standard deviation, KPS: Karnofsky Performance Score, WBRT: Whole Brain Radiation Therapy.

The most common primary cancer types in the progression and RN groups (n=32) included lung (31%), melanoma (31%), and breast (19%), with no significant differences between these groups (p=0.393). Furthermore, no significant differences were found between the RN and progression groups in terms of age, sex, Karnofsky Performance Score, systemic therapies, tumor volume, or dosimetry parameters. Of the entire study population (n=48), immunotherapy was given to 22 (46%), targeted therapy to 20 (42%), and chemotherapy to 17 (35%), while whole-brain radiation therapy was performed in 3 (6%) prior to first SRS. All favorable tumor control cases had a primary cancer diagnosis of NSCLS; 7 (43%) had epidermal growth factor receptor (“EGFR”) mutation.

### GRASP Imaging

Table 2 summarizes the comparison of normalized wash-in and wash-out slopes. Pre-SRS normalized wash-in slope (p=0.616) and wash-out slope (p=0.727) did not significantly differ between RN and TP. Post-SRS normalized wash-in slope was significantly higher in TP than RN on all corresponding follow-up scans (follow-up 1: p=0.021; follow-up 2: p=0.004; follow-up 3: p=0.002). No significant differences in post-SRS normalized wash-out were found between RN and TP. Pre- and post-SRS normalized wash-in and wash-out were not significantly different between the RN and control groups.

TABLE 2
Comparison of normalized wash-in and wash-out slopes in the tumor control
(n = 16), tumor progression (n = 16) and radiation necrosis (n = 16) groups.

| | | Tumor | | | |
|---|---|---|---|---|---|
| Timepoint | Variable | Control | TP | RN | p (TP vs. RN) |
| Baseline | N | 16 | 16 | 16 | |
| | nWin | 0.33 ± 0.17 | 0.31 ± 0.14 | 0.34 ± 0.22 | .616 |
| | nWout | −0.18 ± 0.47 | −0.32 ± 0.71 | −0.23 ± 0.64 | .718 |
| Follow-up 1 | N | 16 | 16 | 16 | |
| | nWin | 0.12 ± 0.04 | 0.29 ± 0.16 | 0.18 ± 0.08 | .021 |
| | nWout | −0.42 ± 0.50 | −0.75 ± 1.14 | −0.34 ± 0.24 | .171 |
| Follow-up 2 | N | 12 | 12 | 16 | |
| | nWin | 0.12 ± 0.04 | 0.35 ± 0.19 | 0.18 ± 0.09 | .004 |
| | nWout | −0.41 ± 0.45 | −1.05 ± 2.56 | −0.59 ± 1.08 | .518 |
| Follow-up 3 | N | 9 | 8 | 13 | |
| | nWin | 0.12 ± 0.05 | 0.32 ± 0.12 | 0.17 ± 0.07 | .002 |
| | nWout | −0.40 ± 0.53 | −1.44 ± 2.14 | −0.57 ± 0.57 | .173 |

Data is described by mean ± standard deviation.

Post-SRS normalized wash-in slope differentiated RN and TP with AUC of 0.74 on scan 1, 0.85 on scan 2, and 0.87 on scan 3. A threshold of 0.18 yielded sensitivity 75% and specificity 69% on scan 1 and sensitivity 92% and specificity 69% on scan 2. A threshold of 0.28 on scan 3 yielded sensitivity 63% and specificity 100%.

### DISCUSSION

A standardized and efficient imaging tool to distinguish tumor progression from radiation-associated or other treatment changes would be of value to clinicians and radiologists. Techniques using standard MRI techniques can be developed to distinguish these diagnostic entities, without requiring the patient to return to the clinic for additional imaging, thus avoiding excess costs and time. Matching can be used on the contrast-enhanced tumor border on T1 images to the T2 defined nodule for brain metastases.

In a longitudinal study it was found that GRASP DCE-MRI has the potential to differentiate between brain-metastasis progression and RN after SRS, using wash-in slope normalized to the superior sagittal sinus as a model-free quantitative measure of tumor tissue enhancement. The study results demonstrated a sensitivity of up to 92% and specificity of up to 100% in differentiating between TP and RN, depending on the timing of post-SRS scans. Tumor tissue enhancement was characterized by rapid wash-in, likely representing a combination of factors related to the hyperdynamic tumor microvasculature such as neo-angiogenesis and vascular permeability, while RN showed a significantly slower wash-in. It is possible that these biological and microvascular processes develop into their unique patterns over time, therefore explaining the increased specificity in the later follow-up scans. In addition, treated tumors that exhibited a favorable response showed slow wash-in. This result remained stable over subsequent scans.

Modalities, such as standard morphological MRI scans, often fail to distinguish TP and RN due to overlapping characteristics such as apparent lesion growth, contrast enhancement, and peri-lesional edema. Many advanced imaging modalities have been studied to aid in this distinction, such as DCE-MRI, DSC-MRI, CT perfusion, PET-CT, and MR spectroscopy. They all have shown variable results and various technical limitations that prevent them from being widely implemented in clinical practice. For example, DSC-MRI, the most commonly used perfusion imaging technique, suffers from limited spatial resolution, effects from recirculation of contrast, and vulnerability to susceptibility artifacts, such as from hemorrhage, calcification, and iatrogenic material, all of which may be present in patients with previously treated brain metastases.

DCE-MRI has the potential to overcome these limitations. Acquired DCE-MRI data is typically processed using multicompartmental pharmacokinetic models, most commonly the Tofts-Kermode model, to derive quantitative parameters such as the influx transfer constant (K^{trans}), the volume of extravascular extracellular space, and the blood plasma volume. However, these models are complex, their usage is often not practical in the routine clinical setting, and they assume that signal-intensity changes directly result from contrast extravasation into the extracellular space without accounting for other factors such as vascular density and tissue composition. In contrast, model-free measures, such as slope and area under the signal-time curve, may be more clinically usable.

A prior study has shown that DCE-MRI can differentiate between TP and RN with a sensitivity of 95% and specificity of 78%, based on the maximum initial slope of enhancement, which was higher in TP. This study can provide complementary results and additional insights due to methodological differences. While the cross-sectional cohort of the prior study mainly consisted of primary brain tumors that underwent surgery, radiation, chemotherapy, or a combination of treatment modalities, the present study utilized a cohort containing only metastatic tumors that specifically underwent SRS and had longitudinal GRASP imaging available for analysis. Additionally, all TP cases in this study had histopathologic confirmation, unlike previous cohorts.

A manner to standardize measurements, particularly across a longitudinal study, is critical for providing useful data. In the embodiment as tested, the analysis utilized a normalized wash-in slope to the superior sagittal sinus as an internal control on each scan to account for variability in scanner acquisition parameters, contrast bolus injection, and patients' hemodynamics. This is notably in contrast to previous analyses that normalized wash-in slope to the peak signal intensity for each tumor ROI.

GRASP imaging offers several advantages over DCE-MRI techniques, both generally and specifically for brain tumor imaging. GRASP's continuous radial sampling and reconstruction process provides high isotropic spatial resolution over the entire brain and adjustable temporal resolution, whereas DCE-MRI techniques often limit the field of view or image resolution to accommodate multiple separate acquisitions. Unlike DCE-MRI, GRASP is robust against artifacts from respiratory motion and arterial pulsation while also providing homogeneous fat suppression, which can aid the differentiation of fat, hemorrhage, and surgical packing in the postoperative setting. Moreover, the continuous data acquisition in GRASP eliminates possible timing errors during the exam, which simplifies clinical implementation and reduces training requirements for technicians.

The incidence of RN after SRS for brain metastasis (“BM”) is estimated between 5 to 25%. The wide variation derives from different definition criteria (imaging or histologically based), increased awareness, and the increased use of routine diagnostic imaging with higher resolution. The major factors thought to cause RN include either radiation-induced vascular damage and disruption of the blood-brain barrier, or direct damage to glial cells and demyelination. The main predisposing risk factors for RN include tumor size and dose, the volume of brain parenchyma receiving >12 Gy, prior radiation exposure, and primary tumor type. The management of RN varies from close follow-up in asymptomatic patients to corticosteroids as first-line treatment in symptomatic cases and then bevacizumab in protracted ones. Refractory cases may require surgical resection of the lesion or laser interstitial thermal therapy. Some clinicians use anticoagulants or hyperbaric oxygen therapy in rare instances. Therefore, accurate, convenient, and non-invasive differentiation of RN from tumor progression is crucial for any neuro-oncology service in order to tailor the appropriate therapeutic approach. GRASP seems to answer these requirements. In addition, unlike methods that require a two-phase test with >1 hour delay, such as the Treatment Response Assessment Maps (“TRAM”)), GRASP offers an immediate analysis by adding a single acquisition of about 6 minutes to the MRI examination. However, further validation of this method in larger studies is needed.

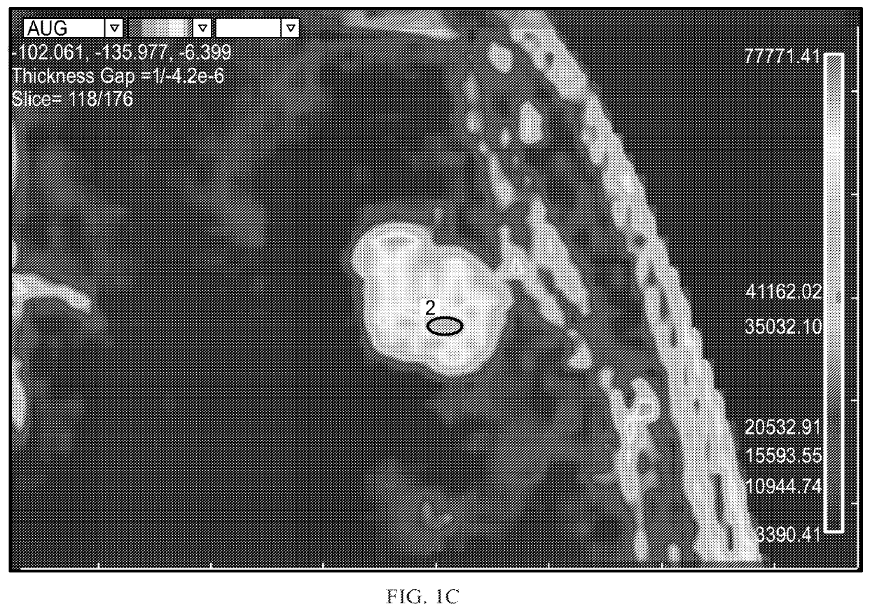

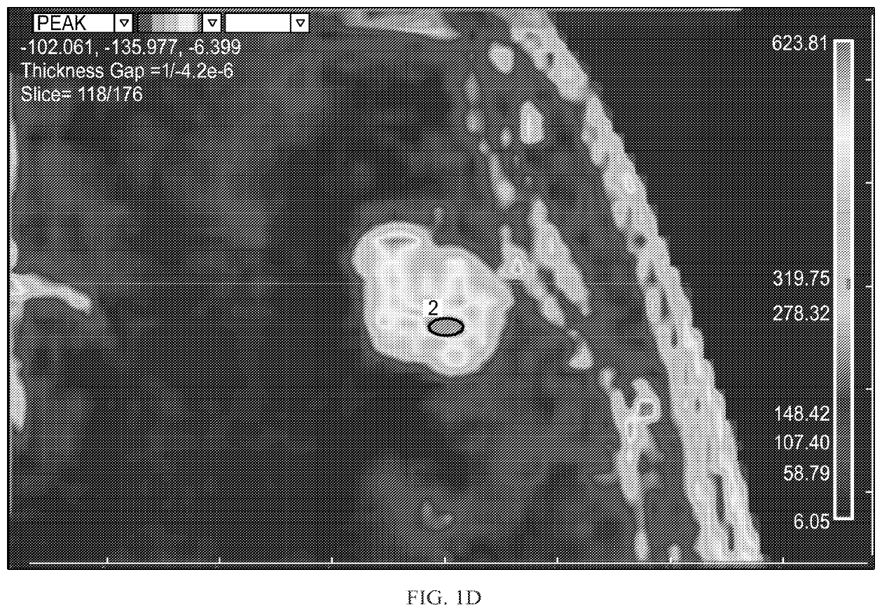

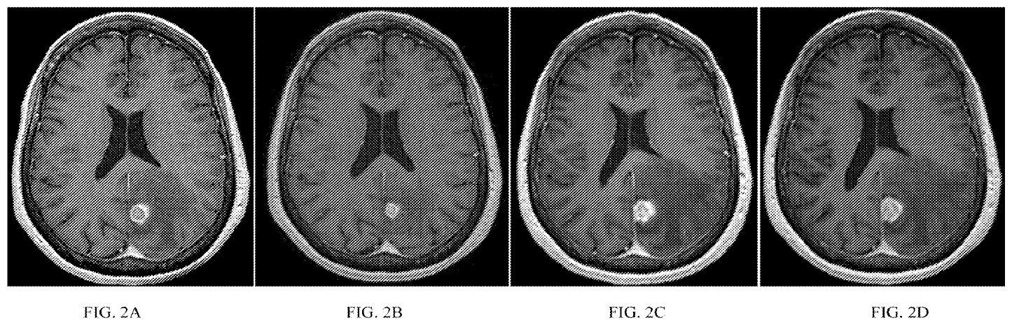

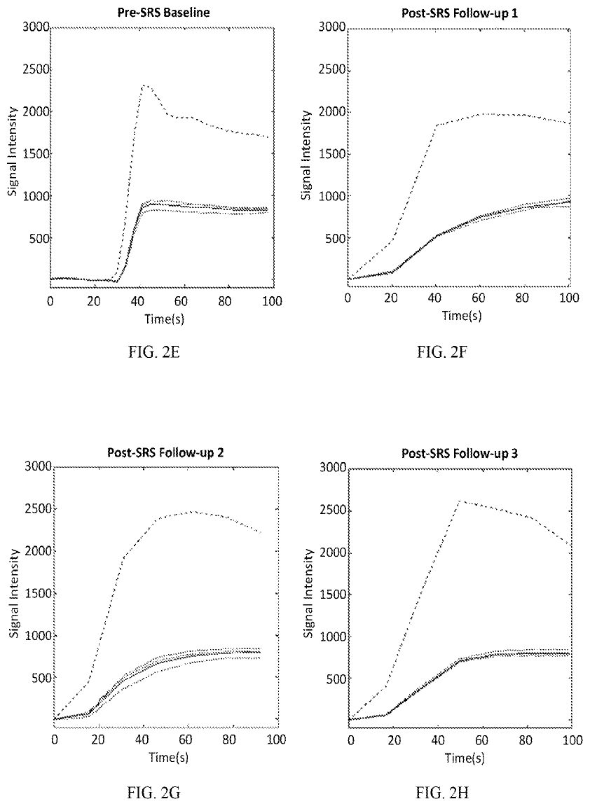

FIGS. 2A-2H illustrate progressive metastasis with signal-time curves. FIGS. 2A-2D are post-contrast T1-weighted images demonstrate an enhancing medial left parietal lobe lesion at baseline on the day of SRS (FIG. 2A), which mildly decreases in size on the first follow-up scan 79 days after SRS (FIG. 2B) but grows on the subsequent scans 139 days (FIG. 2C) and 175 days after SRS (FIG. 2D). FIGS. 2E-2H are signal-time curves show that the wash-in of the tumoral ROIs (dotted) and their averaged signal-time curve (solid) remain high relative to the superior sagittal sinus (dashed). Mean normalized wash-in was 0.38 at baseline (FIG. 2E), 0.31 at follow-up 1 (FIG. 2F), 0.33 at follow-up 2 (FIG. 2G), and 0.40 at follow-up 3 (FIG. 2H). This lesion was resected and pathologically confirmed as viable tumor.

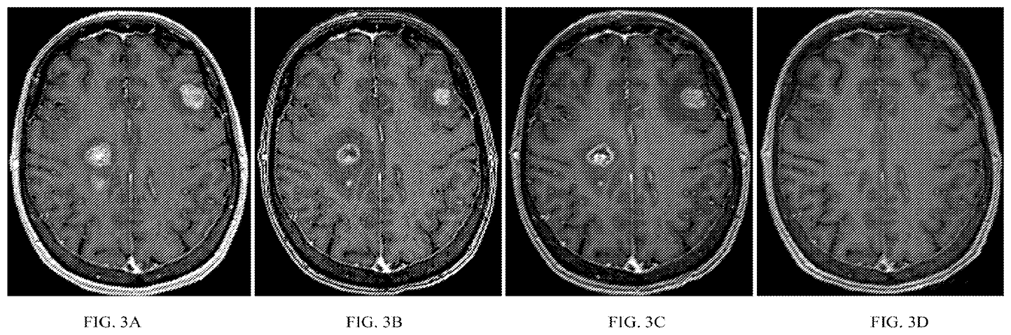

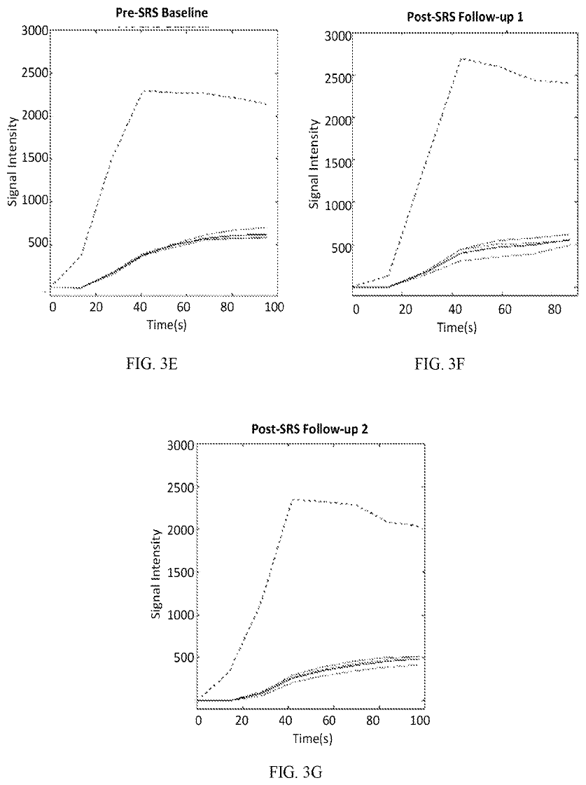

FIGS. 3A-3G illustrate radiation necrosis with signal-time curves. FIGS. 3A-3D are post-contrast T1-weighted images demonstrate a few enhancing lesions in both frontal lobes at baseline on the day of SRS (FIG. 3A), which decrease in size on the first follow-up scan 169 days after SRS (FIG. 3B) and appear larger on the second follow-up scan 272 days after SRS (FIG. 3C). On the third follow-up scan 435 days after SRS, the lesion was nearly resolved with no measurable enhancement (FIG. 3D). FIGS. 3E-3G are signal-time curves corresponding to the lateral left frontal lesion demonstrate low wash-in of tumoral ROIs (dotted) and their average (solid) relative to the superior sagittal sinus (dashed). Mean normalized wash-in was 0.16 on baseline (FIG. 3E), 0.13 on follow-up 1 (FIG. 3F), and 0.15 on follow-up 2 (FIG. 3G).

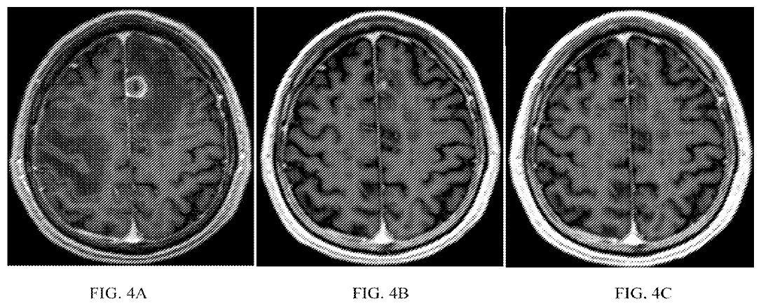

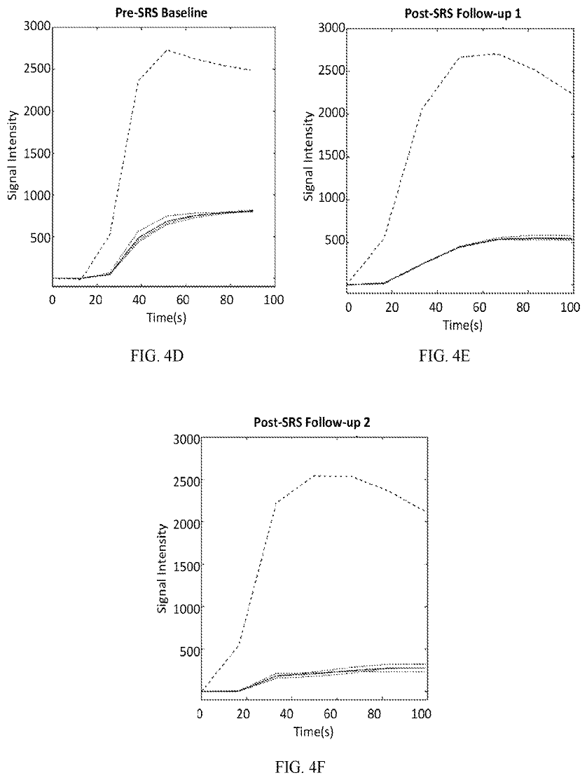

FIGS. 4A-4F illustrate a responsive control tumor with signal-time curves. FIGS. 4A-4C are post-contrast T1-weighted images demonstrate a medial left frontal lobe lesion at baseline 5 days before SRS (FIG. 4A) and as it dramatically shrinks on subsequent scans obtained 155 days (FIG. 4B) and 302 days after SRS (FIG. 4C). FIGS. 4D-4F are mean normalized tumor wash-in (solid) was 0.24 on baseline (FIG. 4D), 0.11 on follow-up 1 (FIG. 4E), and 0.07 on follow-up 2 (FIG. 4F), relative to the superior sagittal sinus (dashed). This lesion nearly completely resolved after another 6 months.

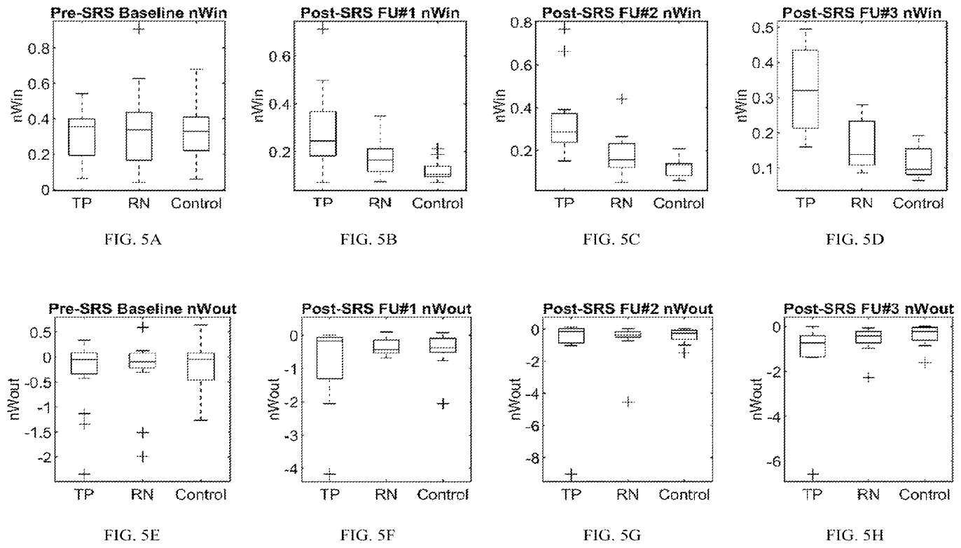

FIGS. 5A-5H illustrate boxplots of normalized wash-in slopes (“nWin,” FIGS. 5A-5D) and normalized wash-out slopes (“nWout,” FIGS. 5E-5H) for tumor progression (“TP”), radiation necrosis (“RN”), and tumor control at baseline and on three post-stereotactic radiosurgery (“SRS”) follow-up scans.

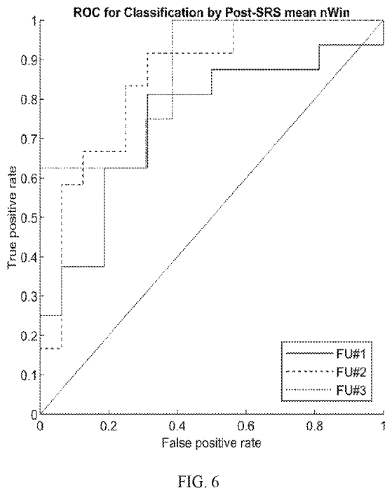

FIG. 6 illustrates ROC curves for the differentiation of tumor progression and radiation necrosis using normalized wash-in slope (“nWin”) on 3 follow-up scans.

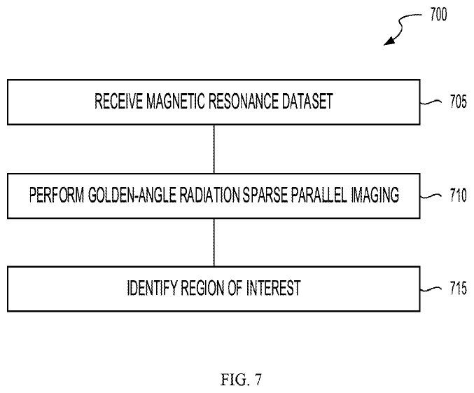

FIG. 7 illustrates a method 700 of magnetic resonance imaging. In brief summary, the method 700 can include receiving a magnetic resonance dataset (BLOCK 705). The method 700 can include performing golden-angle radial sparse parallel (GRASP) imaging (BLOCK 710). The method 700 can include identifying at least one region of interest (BLOCK 715).

The method 700 can include receiving a magnetic resonance dataset (BLOCK 705). The method 700 can include receiving, by at least one processor, a magnetic resonance dataset. The magnetic resonance dataset can include at least one scan.

The method 700 can include performing golden-angle radial sparse parallel imaging (BLOCK 710). The method 700 can include performing, by the at least one processor, golden-angle radial sparse parallel imaging on the magnetic resonance dataset. For example, the method 700 can include performing golden-angle radial sparse parallel imaging on the magnetic resonance dateset to output one or more images.

The method 700 can include identifying at least one region of interest (BLOCK 715). The method 700 can include identifying, by the at least one processor, at least one region of interest in the one or more images. The at least one region of interest can correspond to at least one of tumor progression (e.g., brain-metastasis progression) or radiation effects (e.g., radiation necrosis). Radiation effects can include adverse radiation effects or undesirable radiation effects. Radiation effects can include radiation necrosis (RN).

The at least one region of interest can correspond to tumor progression. The at least one region of interest corresponding to tumor progression can have a faster wash-in than a region of interest corresponding to radiation effects.

The at least one region of interest can correspond to radiation effects. The radiation effects can include radiation necrosis. The at least one region of interest corresponding to radiation necrosis can have a slower wash-in than a region of interest corresponding to tumor progression.

The method 700 can include extracting, by the at least one processor, one or more signal-time curves. The method 700 can include calculating, by the at least one processor, a slope of the one or more signal-time curves during wash-in. Wash-in can include a period of maximally rapid increasing superior sagittal sinus signal. Wash-in can include an influx of contrast. The method 700 can include calculating, by the at least one processor, a slope of the one or more signal-time curves during wash-out. Wash-out can include a period of monotonically decreasing signal intensity after peak superior sagittal sinus enhancement.

The method 700 can include normalizing, by the at least one processor, a wash-in slope of the at least one region of interest. For example, the method 700 can include normizing the wash-in slope to the superior sagittal sinus as a model-free quantitative measure of tumor tissue enhancement.

The method 700 can include normalizing, by the at least one processor, a wash-out slope of the at least one region of interest. For example, the method 700 can include normizing the wash-out slope to the superior sagittal sinus as a model-free quantitative measure of tumor tissue enhancement.

The method 700 can include differentiating, by the at least one processor, the at least one region of interest between tumor progression and radiation effects. For example, the method 700 can include distinguishing, by the at least one processor, between tumor progression and radiation effects. The method 700 can include distinguishing, by the at least one processor, the at least one region of interest between tumor progression and radiation effects. For example, the method 700 can include categorizing the region of interest at one of tumor progression or radiation effects (e.g., radiation necrosis).

The method 700 can include differentiating, by the at least one processor, the at least one region of interest between tumor progression and radiation effects with a sensitivity of greater than 90%. For example, the method 700 can include differentiating, by the at least one processor, the at least one region of interest between tumor progression and radiation effects with a sensitivity of 92%.

The method 700 can include differentiating, by the at least one processor, the at least one region of interest between tumor progression and radiation effects with a specificity of greater than 90%. For example, the method 700 can include differentiating, by the at least one processor, the at least one region of interest between tumor progression and radiation effects with a specificity of 100%.

Those of skill in the art will appreciate that the embodiments should not be limited by the limitations of the example study, including its relatively small size in specific cancer types and potential confounding effects of different systemic agents used to treat the primary cancer.

A system (e.g., MRI system) can include at least one processor. The system can include a memory, with computer code instructions stored thereon. The computer code instructions, when executed by the at least one processor, can cause the at least one processor to receive a magnetic resonance dataset including at least one scan. The computer code instructions, when executed by the at least one processor, can cause the at least one processor to perform golden-angle radial sparse parallel imaging on the magnetic resonance dataset to output one or more images. The computer code instructions, when executed by the at least one processor, can cause the at least one processor to identify at least one region of interest in the one or more images, the at least one region of interest corresponding to at least one of tumor progression or radiation effects

The computer code instructions, when executed by the at least one processor, can cause the at least one processor to extract one or more signal-time curves. The computer code instructions, when executed by the at least one processor, can cause the at least one processor to calculate a slope of the one or more signal-time curves during wash-in. The computer code instructions, when executed by the at least one processor, can cause the at least one processor to calculate a slope of the one or more signal-time curves during wash-out.

The computer code instructions, when executed by the at least one processor, can cause the at least one processor to normalize a wash-in slope of the at least one region of interest. The computer code instructions, when executed by the at least one processor, can cause the at least one processor to normalize a wash-out slope of the at least one region of interest.

The computer code instructions, when executed by the at least one processor, can cause the at least one processor to differentiate the at least one region of interest between tumor progression and radiation effects. The computer code instructions, when executed by the at least one processor, can cause the at least one processor to differentiate the at least one region of interest between tumor progression and radiation effects with a sensitivity of greater than 90%. The computer code instructions, when executed by the at least one processor, can cause the at least one processor to differentiate the at least one region of interest between tumor progression and radiation effects with a specificity of greater than 90%. The computer code instructions, when executed by the at least one processor, can cause the at least one processor to differentiate the at least one region of interest between tumor progression and radiation effects with a specificity of 100%.

The at least one region of interest can correspond to tumor progression. The at least one region of interest corresponding to tumor progression can have a faster wash-in than a region of interest corresponding to radiation effects.

The at least one region of interest can correspond to radiation effects. The radiation effects can include radiation necrosis. The at least one region of interest corresponding to radiation necrosis can have a slower wash-in than a region of interest corresponding to tumor progression

### Definitions

No claim element herein is to be construed under the provisions of 35 U.S.C. § 112(f), unless the element is expressly recited using the phrase “means for.”

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Any references herein to the positions of elements (e.g., “top,” “bottom,” “above,” “below”) are merely used to describe the orientation of various elements in the figures. It should be noted that the orientation of various elements may differ according to other exemplary embodiments, and that such variations are intended to be encompassed by the present disclosure.

Embodiments of the subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices).

The operations described in this specification can be performed by a data processing apparatus on data stored on one or more computer-readable storage devices or received from other sources. The term “data processing apparatus” or “computing device” encompasses various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a circuit, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more circuits, subprograms, or portions of code). A computer program can be deployed to be executed on one computer or on multiple computers that are located at one site or distributed across multiple sites and interconnected by a communication network.

Processors suitable for the execution of a computer program include, by way of example, microprocessors, and any one or more processors of a digital computer. A processor can receive instructions and data from a read only memory or a random access memory or both. The elements of a computer are a processor for performing actions in accordance with instructions and one or more memory devices for storing instructions and data. A computer can include, or be operatively coupled to receive data from or transfer data to, or both, one or more mass storage devices for storing data, e.g., magnetic, magneto optical disks, or optical disks. A computer need not have such devices. Moreover, a computer can be embedded in another device, e.g., a personal digital assistant (PDA), a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The implementations described herein can be implemented in any of numerous ways including, for example, using hardware, software or a combination thereof. When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may comprise a memory, one or more processing units (also referred to herein simply as “processors”), one or more communication interfaces, one or more display units, and one or more user input devices. The memory may comprise any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to or receive communications from other devices. The display unit(s) may be provided, for example, to allow a user to view various information in connection with execution of the instructions. The user input device(s) may be provided, for example, to allow the user to make manual adjustments, make selections, enter data or various other information, or interact in any of a variety of manners with the processor during execution of the instructions.

The various methods or processes outlined herein may be coded as software that is executable on one or more processors that employ any one of a variety of operating systems or platforms. Additionally, such software may be written using any of a number of suitable programming languages or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the solution discussed above. The computer readable medium or media can be transportable, such that the program or programs stored thereon can be loaded onto one or more different computers or other processors to implement various aspects of the present solution as discussed above.

The terms “program” or “software” are used herein to refer to any type of computer code or set of computer-executable instructions that can be employed to program a computer or other processor to implement various aspects of embodiments as discussed above. One or more computer programs that when executed perform methods of the present solution need not reside on a single computer or processor, but may be distributed in a modular fashion amongst a number of different computers or processors to implement various aspects of the present solution.

Computer-executable instructions may be in many forms, such as program modules, executed by one or more computers or other devices. Program modules can include routines, programs, objects, components, data structures, or other components that perform particular tasks or implement particular abstract data types. The functionality of the program modules can be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Various embodiments are described in the general context of method steps, which may be implemented in one embodiment by a program product including computer-executable instructions, such as program code, executed by computers in networked environments. Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps.

Software and web implementations of the present invention could be accomplished with standard programming techniques with rule based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

As used herein, the singular forms “a”, “an” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A method, comprising:
receiving, by at least one processor, a magnetic resonance dataset comprising at least one scan;
performing, by the at least one processor, golden-angle radial sparse parallel (GRASP) imaging on the magnetic resonance dataset to output one or more images; and
identifying, by the at least one processor, at least one region of interest in the one or more images, the at least one region of interest corresponding to at least one of tumor progression or radiation effects.

2. The method of claim 1, further comprising extracting, by the at least one processor, one or more signal-time curves.

3. The method of claim 1, further comprising:
extracting, by the at least one processor, one or more signal-time curves; and
calculating, by the at least one processor, a slope of the one or more signal-time curves during wash-in.

4. The method of claim 1, further comprising:
extracting, by the at least one processor, one or more signal-time curves; and
calculating, by the at least one processor, a slope of the one or more signal-time curves during wash-out.

5. The method of claim 1, further comprising normalizing, by the at least one processor, a wash-in slope of the at least one region of interest.

6. The method of claim 1, further comprising normalizing, by the at least one processor, a wash-out slope of the at least one region of interest.

7. The method of claim 1, further comprising differentiating, by the at least one processor, the at least one region of interest between tumor progression and radiation effects.

8. The method of claim 1, further comprising differentiating, by the at least one processor, the at least one region of interest between tumor progression and radiation effects with a sensitivity of greater than 90%.

9. The method of claim 1, further comprising differentiating, by the at least one processor, the at least one region of interest between tumor progression and radiation effects with a specificity of greater than 90%.

10. The method of claim 1, further comprising differentiating, by the at least one processor, the at least one region of interest between tumor progression and radiation effects with a specificity of 100%.

11. The method of claim 1, wherein:
the at least one region of interest corresponds to tumor progression; and
the at least one region of interest corresponding to tumor progression has a faster wash-in than a region of interest corresponding to radiation effects.

12. The method of claim 1, wherein the at least one region of interest corresponds to radiation effects, the radiation effects comprising radiation necrosis.

13. The method of claim 1, wherein:
the at least one region of interest corresponds to radiation effects, the radiation effects comprising radiation necrosis; and
the at least one region of interest corresponding to radiation necrosis has a slower wash-in than a region of interest corresponding to tumor progression.

14. A system, comprising:
at least one processor;
a memory, with computer code instructions stored thereon, the computer code instructions, when executed by the at least one processor, cause the at least one processor to:
receive a magnetic resonance dataset comprising at least one scan;
perform golden-angle radial sparse parallel (GRASP) imaging on the magnetic resonance dataset to output one or more images; and
identify at least one region of interest in the one or more images, the at least one region of interest corresponding to at least one of tumor progression or radiation effects.

15. The system of claim 14, wherein:
the computer code instructions, when executed by the at least one processor, cause the at least one processor to:
extract one or more signal-time curves; and
calculate a slope of the one or more signal-time curves during wash-in.

16. The system of claim 14, wherein:
the computer code instructions, when executed by the at least one processor, cause the at least one processor to:
extract one or more signal-time curves; and
calculate a slope of the one or more signal-time curves during wash-out.

17. The system of claim 14, wherein:
the computer code instructions, when executed by the at least one processor, cause the at least one processor to:
normalize at least one of a wash-in slope or a wash-out slope of the at least one region of interest.

18. The system of claim 14, wherein:
the computer code instructions, when executed by the at least one processor, cause the at least one processor to:
differentiate the at least one region of interest between tumor progression and radiation effects.

19. The system of claim 14, wherein:
the at least one region of interest corresponds to tumor progression; and
the at least one region of interest corresponding to tumor progression has a faster wash-in than a region of interest corresponding to radiation effects.

20. The system of claim 14, wherein:
the at least one region of interest corresponds to radiation effects, the radiation effects comprising radiation necrosis.
